(19) United States
(12) Patent Application Publication
(10) Pub. No.: US 2009/0163841 A1
(43) Pub. Date: Jun. 25, 2009

(54) Lumbar Adjustment System

(75) Inventor: Geoffrey Garth, Long Beach, CA (US)

Correspondence Address: FISH & ASSOCIATES, PC;ROBERT D. FISH, 2603 Main Street, Suite 1000, Irvine, CA, 92614-6232, US

(73) Assignee: ASPEN MEDICAL PRODUCTS, INC., Irvine, CA (US)

(21) Appl. No.: 11/960,614

(22) Filed: Dec. 19, 2007

(51) Int. Cl. A61F 5/00

(52) U.S. Cl. 602/19

(57) ABSTRACT

A lumbar support includes a flexible lumbar piece mechanically coupled to a back panel, and a rotatable adjusting member, preferably a dial accessible by the wear, that adjusts the extent the lumbar piece bows away from the back panel. A series of detents can be used to provide discrete “stops”. Lumbar pieces can comprise any suitable material, including for example metal or plastic, and are preferably removable from the back panel.

### FIELD OF THE INVENTION

The field of the invention is orthotics (class 602/005).

### BACKGROUND

Numerous orthotic devices are known, many of which provide adjustments to conform to the anatomy of a wearer. Different girths are relatively easy to accommodate using one or more belts, straps, frames, and in some cases lacings. Different lordoses are somewhat more difficult to accommodate because lordotic curves vary in length, depth, and vertical positioning relative the pelvis and waist.

One solution is to provide a back panel with replaceable lordosis forms. U.S. Pat. No. 7,186,229 to Schwenn et al. (Mar. 2007), for example, teaches a back pocket that can receive different “stays”. While in theory the use of a wide range of different forms could accommodate all variations in anatomy, it is impractical for a medical provider to carry a sufficiently large stock of forms.

Another solution is to use a form that can be bent into a desired shape by a practitioner. U.S. Pat. No. 6,964,644 to Garth (Nov. 2005), for example, teaches a malleable metal frame used to accommodate different sacral curvatures. Although malleable forms can theoretically be bent into any suitable shape, the practicality is sometimes wanting. Among other things, practitioners differ widely in the level of skill that can be consistently applied to bending the forms.

Still a third solution is to fit the orthotic with rods or other adjustment mechanisms that bend a back panel. U.S. Pat. No. 6,702,770 to Bremer et al. (Mar. 2004) teaches such a device. In that patent a preferred adjustment mechanism includes a bolt extending between the upper and lower regions of the back panel, which can be tightened or loosened against a nut by turning the bolt with a screwdriver. It is not at all clear, however, that rotatable adjustment mechanisms requiring a tool are desirable for lower torso or hip orthotic. The claimed Bremer orthotic, for example, does not appear to have been particularly successful in the marketplace.

Yet another solution is to incorporate inflatable bladders within an orthotic as described in U.S. Pat. No. 5,950,628 to Dunfee (Sep. 1999). Although inflatable bladders are easy to control, they do not necessarily conform to a desired supporting shape. Another disadvantage for the wearer is the bladders tend to be bulky.

Rotating lordosis adjustments are known for chairs. U.S. Pat. No. 6,079,783 to Schuster, Sr., et al, (June 2000), for example, teaches a lordosis adjustment that is manipulated by turning a knob on either side of the chair. The knobs operate a cable and tension spring that cooperate to adjust the degree to which a panel bows out from the chair frame. U.S. Pat. No. 5,335,965 to Sessini (August 1994) teaches an adjustable cushion for chairs that utilizes an elastic plate that is adjusted through a rotating, cylindrical threaded scroll. Another example is taught in U.S. Pat. No. 5,461,205 to Schmnidt (June 1997). The aforementioned patents, and all other extrinsic materials discussed herein are incorporated by reference in their entirety. Where a definition or use of a term in an incorporated reference is inconsistent or contrary to the definition of that term provided herein, the definition of that tenn provided herein applies and the definition of that term in the reference does not apply.

There are considerable technical difficulties in applying an adjustment knob (or dial, which is considered to be a flat knob) to an orthotic. Indeed, the only device to accomplish that in a commercially viable manner is described in US 2007/0027418 to Calco (publ. Feb. 2007). In that case a cervical collar is geared, and adjusted using a knob positioned near the chin. A similarly convenient space to locate an adjusting knob is unavailable in a lower torso or hip orthotic.

Thus, there is still a need to provide an orthotic having an adjustable member that adjusts the bowing of a lumbar piece. Further, it is advantageous to provide a user the ability to adjust a lumbar piece without requiring a tool and to have the lumbar piece adjust to the lordosis that it finds to insure broad contact.

### SUMMARY OF THE INVENTION

The present invention provides apparatus, systems and methods in which an orthotic includes a flexible lumbar piece mechanically coupled to a back panel, and an adjusting member that is operatively positioned to alter bowing of the lumbar piece relative to the back panel.

The lumbar piece is structural, as opposed for example to being a foam liner or cushion. In preferred embodiments the lumbar piece comprises a metal or plastic, and is removable from the back panel. A dial or lever can advantageously be used to rotate a rack and pinion or other adjustment mechanism to bow the lumbar pieces as desired. A series of detents can be used to provide discrete “stops”.

For the purposes of this document, the term “orthotic” or “orthosis”, as used herein means a wearable orthopedic appliance or apparatus used to support, align, prevent or correct deformities or to improve the function of movable parts of the body.

Various objects, features, aspects and advantages of the inventive subject matter will become more apparent from the following detailed description of preferred embodiments, along with the accompanying drawings in which like numerals represent like components.

### BRIEF DESCRIPTION OF THE DRAWING

FIG. 1A is a front view of a preferred orthotic, showing a lumbar piece coupled to a back panel.

FIG. 1B is a back view of a preferred orthotic from FIG. 1, showing a lordotic adjustment knob.

FIG. 1C is a front view of a preferred orthotic from FIG. 1A, showing an extension piece that is used to increase the effective length of the lumbar piece.

FIG. 2A is an internal, front view schematic of an example embodiment of an adjustment mechanism by which rotation of a dial changes the bowing of the lumbar piece.

FIG. 2B is an internal, front view schematic of an alternative example embodiment of an adjustment mechanism from FIG. 2A by which rotation of a lever changes the bowing of the lumbar piece.

### DETAILED DESCRIPTION

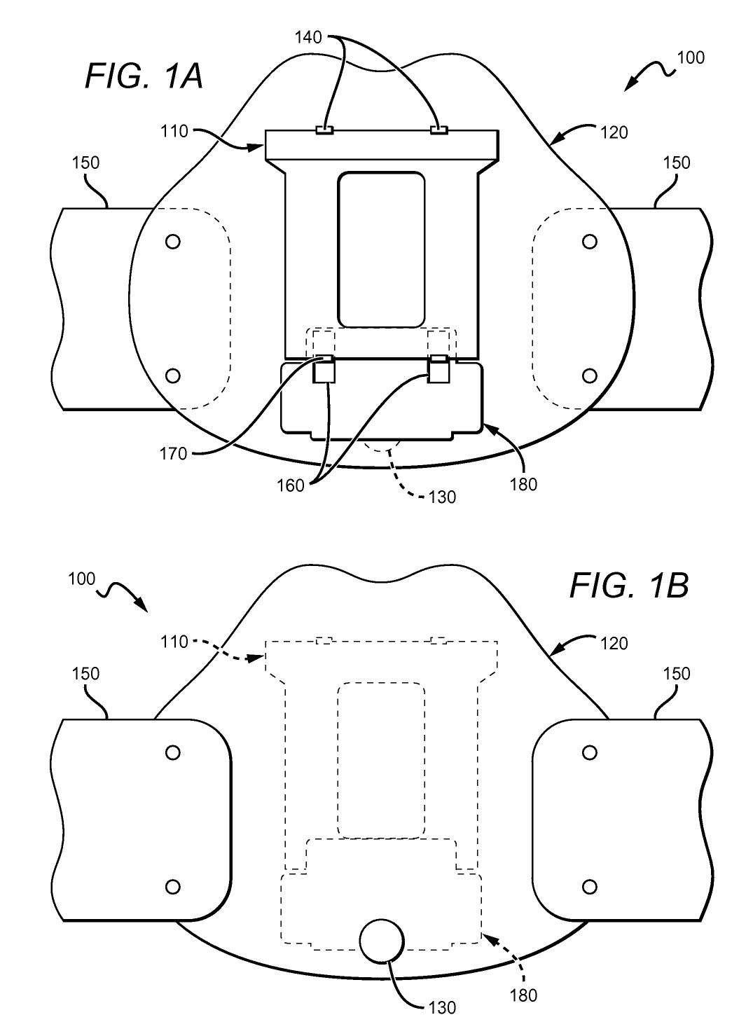

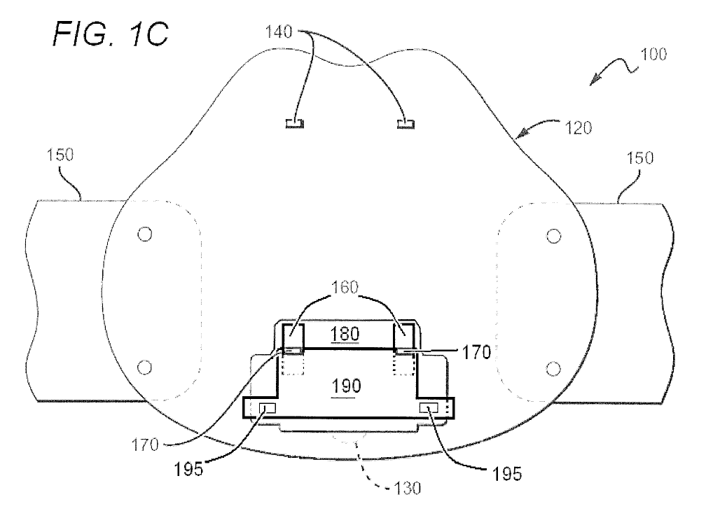

FIG. 1A represents a front view of a preferred orthotic showing a lumbar piece coupled to a back panel. Orthotic 100 generally comprises a back panel 120, a lumbar piece 110, and a knob 130 coupled to adjustment mechanism 180. A user wears orthotic 100 by placing back panel 120 against the lumbar region of their lower torso in manner where lumbar piece 110 is able to apply pressure to the user's back. As knob 130 rotates, adjustment mechanism 180 responds by moving adjustment slider coinector 170 within slots 160 which in turn push against lumbar piece 110. The user of orthotic 100 is, therefore, able to adjust the amount of contact provided by lumbar piece 110 by rotating knob 130.

Back Panel

Back panel 120 provides a rigid structure for orthotic 100. In a preferred embodiment, back panel 120 couples to one or more straps 150 representing an additional member of oithotic 100. Straps 150 in combination with back panel 120 allow orthotic 100 to girdle the lower torso of an individual wearing orthotic 100 or otherwise position back panel 120 against an individual's back. One skilled in the art of orthotics will appreciate that strap 150 is used here euphemistically to represent any type of additional member, including for example, a corset, a belt, an elastic band, a brace, a frame, a lacing or other strapping system.

Once an individual puts on orthotic 100 and straps 150 are suitably tightened, the individual adjusts lumbar piece 110 by rotating knob 130 causing lumbar piece 110 to bow (i.e. flex) away from back panel 120. The combination of straps 150 and back panel 120 counters the pressure from lumbar piece 110 resulting in lumbar piece 110 pressing against the paraspinal muscles in the patient's lumbar region.

In a preferred embodiment, back panel 120 contours to the curves associated with the back of an individual and is formed from a suitably rigid material. Contemplated materials include plastic, metal, or other material that can form desired contours. Preferred embodiments employ injection molded plastic to reduce costs of manufacture while maintaining a substantially rigid support for orthotic 100.

Lumbar Piece

Lumbar piece 110 mechanically couples to back panel 120 in a manner that allows lumbar piece 110 to bow away from back panel 120, and to flatten or relax toward back panel 120. In a preferred embodiment, an upper edge of lumbar piece 110 couples to back panel 120 by connecting to one or more of connector 140 so that the upper edge does not substantially move relative to back panel 120 as lumbar piece 110 is adjusted. A preferred connector includes a slot that receives an upper surface of lumbar piece 110. Preferably, lumbar piece 110 connects to connectors 140 without requiring a tool. In another embodiment, lumbar piece 110 is molded to have posts at the upper corners of lumbar piece 110 that insert into sockets (connector 140) coupled to or molded into back panel 120. Alternative connectors include Velcro®, snaps, sockets, or other fasteners that do not require tools.

A lower part (e.g. an edge, comner, or other surface) of lumbar piece 110 couples to one or more of adjustment slider connector 170. Preferably connector 170 comprises a socket and post assembly, where a post of lumbar piece 110 slides into a socket within slider connector 170. As slider connector 170 moves tip or down within slots 160 relative to back panel 120, the lower edge of lumbar piece 110 also moves. Because the top edge of lumbar piece is preferably fixed relative to back panel 120, lumbar piece 110 will bow as the lower edge moves. When the lower edge is moved toward the upper edge, lumbar piece 110 will tend to bow away from back panel 120 toward the wearer's back. When the lower edge is moved away from the upper edge, lumbar piece 110 flattens or relaxes toward back panel 120 away from the wearer's back. One should appreciate that the described motion of lumbar piece 110 presented above is a single example and that alternatives are also possible. For example, the lower edge of lumbar piece 110 could be fixed relative to back panel 120 while the upper edge moves (not shown). All contemplated motions resulting in bowing of lumbar piece 110 fall within the scope of the inventive subject matter.

Although lumbar piece 110 is represented a single unit within FIG. 1, it is contemplated that lumbar piece 110 could comprise one or more units. For example, in an alternative embodiment (not shown), rather than having a single plate functioning as lumbar piece 110, two plates could be used. The two plates could advantageously straddle the spine, and operate independently to create differential right and left side supports for the lumbar region.

Lumbar piece 110 preferably is fashioned in manner to apply pressure to either side of the wearer's spine for comfort. Additionally, each individual unit of the multiple units composing lumbar piece 110 can be comprise of different material, potentially with different spring constants, to further tune how lumbar piece 110 benefits a wearer.

In especially preferred embodiments, lumbar piece 110 includes a gap or hole allowing the lateral sides of lumbar piece straddle the wearer's spine. A gap or hole advantageously improves a wearer's experience by reducing or eliminating pressure directly on the wearer's spine. One skilled in orthotics will recognize additional features of lumbar piece 110 are also possible including padding for comfort, lateral slides for better position near a spine, or other beneficial features.

In a preferred embodiment, lumbar piece 110 comprises a flexible plate with a suitable spring constant. Example plates include those comprised of metal, plastic, or other materials including capable of bowing under stress or relaxing when the stressor is removed. Additionally, in a preferred embodiment, lumbar piece 110 is replaceable with a different lumbar piece allowing for fine tuning of orthotic 100 to meet the requirements of the individual. Individual requirements include size of the individual, comfort level, required support pressure, length, depth of curvature, or vertical positioning. As an example, consider a child that requires orthotic 100. The child might require lumbar piece 110 to be more flexible than required by an adult; therefore, a lumbar piece having a smaller spring constant would be required. Alternatively, a child might require a smaller effective length of the lumbar piece.

Orthotic 100 is preferably adapted to allow lumbar piece 110 to be removed or replaced as desired. In a preferred embodiment, lumbar piece 110 can be inserted into back panel 120 by flexing lumbar piece 110 and inserting its upper edge into slotted connector 140 and inserting its lower edge into slotted slider connector 170. Tension within lumbar piece 110 holds the piece in position. Conversely, lumbar piece 110 can be removed from orthotic 100 by bowing the piece and removing its upper and lower edges from connectors 140 and 170.

In the exemplary embodiment of an adult sized orthotic shown in FIG. 1A, lumbar piece 110 has an effective length of at least 17 cm. Effective length is defined herein to mean the surface distance between contact points of the material being bowed. In other contemplated embodiments the effective length is between 5 cm and 30 cm. Unless the context dictates otherwise, all ranges herein should be interpreted as being inclusive of their endpoints. It is contemplated the effective length of lumbar piece 110 can be adjusted by changing positions of connectors 140, 170 or 195, or by changing lumbar piece 110 to insure it has maximum useful contact with the wearer's lumbar region.

In more preferred embodiments the length of lumbar piece 110 can be increased by providing one or more additional pieces to orthotic 100. For example, FIG. 1C represents a front view of orthotic 100 from FIG. 1A including extension piece 190 that allows for lumbar piece 110 to have a greater length. Slider 190 couples to connector 170 and also provides one or more of connector 195 to which lumbar piece 110 (not shown) connects. Slider 190 is sized and dimensioned in a manner where connectors 195 are preferably placed near the bottom of back panel 120, lower than connectors 170. One should note the length as measured from connectors 140 to connectors 195 is greater than the length from connectors 140 to connectors 170. By having connectors 195 placed lower than connectors 170, the length of lumbar piece 110 can be increased which can result in a greater effective length.

It is also contemplated orthotic 100 could comprise modular parts for ease of configuration or replacement.

Knob

FIG. 1B is a back view of a preferred orthotic from FIG. 1, showing a lordotic adjustment knob. Knob 130 represents an adjusting member mechanically coupled to lumbar piece 110. In a preferred embodiment orthotic 100 is adapted to covert the rotational motion of knob 130 into linear motion of the lower edge of lumbar piece 110 via adjustment mechanism 180. Orthotic 100 can be adjusted by rotating knob 130 clockwise or counter clockwise causing lumbar piece 110 to bow toward the wearer's spine or to flatten toward pack panel 120. In preferred embodiment, knob 130 resides near the lower portion of orthotic 100 near the lower edge of lumbar piece 110 to provide access to the wearer for adjusting orthotic 100.

It is contemplated that alternative embodiments can employ multiple knobs (not shown) to control or configure the orthotic. For example, in embodiments where the lumbar piece comprises multiple plates, each plate could have separate rotatable adjusting members (not shown). All other orthotic controls also fall within the scope of the inventive subject matter.

Knob 130 can rotate continuously or discretely, thereby causing lumbar piece 110 to be position either continuously or discretely. In some embodiments of knob 130 employ a series of detents (not shown) that provide discrete positions where knob 130 substantially locks into a position preventing lumbar piece 110 from flattening and ensures lumbar piece 110 is also substantially locked into a desired position. The discrete positions could also be locked through a ratchet and pawl assembly. Alternative embodiments of knob 130 are contemplated that would allow the knob to rotate continuously, thus allowing a wearer to configure the lumbar piece into nearly any desired setting. In that case, knob 130 can be locked into position using a contemplated lock control.

Adjustment Mechanism: Example

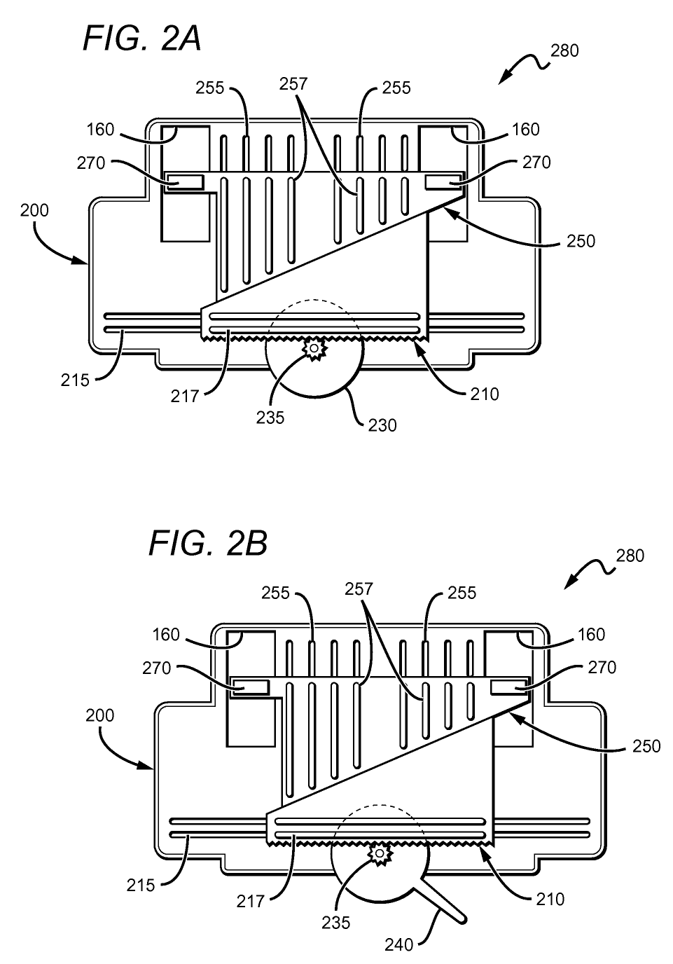

FIG. 2A is an internal, front view schematic of an example embodiment of an adjustment mechanism by which rotation of a dial changes the bowing of the lumbar piece. In FIG. 2A, knob 130 from FIG. 1A and 1B is embodied by dial 230 which represents the adjusting member of orthotic 100. In one embodiment, adjustment mechanism 280 comprises housing 200 that contains parts that respond to rotating dial 230. Dial 230 couples to pinion 235 which in turn couples to slider 250. Slider 250 comprises slider connectors 270 that fit within slots 260 of housing 200. In addition, slots 260 can correspond to slots 160 in some embodiments where housing 200 is attached directly to back panel 120. It is also contemplated that housing 200 can be modeled into back panel 120.

In the example embodiment of FIG. 2A, as dial 230 rotates pinion 235 also rotates. In preferred embodiments, dial 230 and pinion 235 are formed as a single unit. When pinion 235 rotates clockwise, rack 210 will shift to the right relative to back panel 120. Rack 210 is adapted with one or more of rail 217 that fit within one or more of horizontal track 215 placed within the wall of housing 200. Rail 217 and track 215 prevent rack 210 from moving up and down relative to back panel 120. Conversely when pinion 235 rotates counterclockwise, rack 210 will shift to the left relative to back panel 120 along track 215. In a preferred embodiment, rack 210 is advantageously shaped to cause slider 250 to move up and down relative to back panel 120 as rack 210 shifts left and right. Slider 250 can include one or more of rail 257 that fit within one or more vertical track 255 which prevent slider 250 from moving left and right relative to back panel 120. For example, rack 210 and slider 250 can be shaped in a substantially triangular shape and arranged in the manner shown in FIG. 2A. Rack 210 moves left and right, the resulting motion forces slider 250 to move up and down due to their contacting, angled edges.

It is also contemplated that the dial 230 or pinion 235 can couple to a spring mechanism (not shown) within housing 200 to prevent accidental repositioning of a lumbar piece. For example, the spring would apply a sufficient downward force against dial 230 to disengage pinion 235 from rack 210. When an individual wishes to rotate dial 230, they would apply pressure, preferably an upward pressure, to dial 230 countering the force of the spring mechanism. Dial 230 would then engage pinion 235 with rack 210 to allow the lumbar piece to be adjusted. Additionally, it is contemplated that rack 210 can rest on a set of fixed “teeth”, possibly molded into the bottom of housing 200, when pinion 235 is disengaged. Under such conditions, the force of the lumbar piece or the spring would firmly hold rack 210 against the teeth preventing lumbar piece from relaxing or otherwise changing position.

In some embodiments, as shown in FIG. 2A, slider connector 270 corresponds to adjustment slider connector 170 of FIG. 1B which couples to lumbar piece 110. In other embodiments, elements associated with adjustment mechanism 280 can be added or changed for various benefits. For example, additional sliding plates can be added to gain greater movement or additional gears can be added to gain mechanical leverage.

FIG. 2B is an internal, front view schematic of an alternative example embodiment of an adjustment mechanism 280 by which rotation of a lever changes the bowing of the lumbar piece. Leveraging the modularity of adjustment mechanism 280, changing dial 230 to lever 240 could offer an individual greater leverage over bowing lumbar piece 110. One skilled in the art will appreciate there are numerous alternative embodiments where an adjustment mechanism adjusts the bowing of lumbar piece 110 without requiring a tool. One contemplated alternative example includes, but is not limited to, a cable system allowing a user to pull cables causing lumbar piece 110 to bow through sliding plates within the adjustment mechanism or through rotating a worm-screw shaft. Additional contemplated examples include a post system coupled to lumbar piece 110 where a user moves the post up or down to adjust the bowing of lumbar piece 110, or a lateral sliding panel system where a user manually slides panels similar to those as described in adjustment mechanism 280.

All contemplated adjustment mechanisms capable of bowing lumbar piece 110 without requiring a tool fall within the scope of the inventive subject matter. For example, a handle could be moved laterally causing the lumbar piece to increase or decrease the bowing of lumbar piece 110.

Preferred embodiments comprise an adjustment mechanism 280 that is substantially thin or compact to fit closely to back panel 120. Additional contemplated features of adjustment mechanisms include modular mechanisms where adjustment mechanism 280 can be inserted or removed from back panel 120 or where adjustment mechanism can be disassembled, fixed, or reassembled without requiring a tool.

Advantages

Those skilled in the art will appreciate the numerous advantages of the presented subject matter. One advantage of the disclosed subject matter is that the orthotic is relatively thin due to the compact structure of the lumbar adjusting mechanism as opposed to bulky, inflatable orthotics. Unlike inflatable bladders which exert pressure on the spine as well as the musculature, the disclosed mechanism can be designed to apply pressure only in the areas where it is desired. It also has the advantage that, unlike bladder systems, it does not move and shift as the patient moves. Another advantage is the wearer of the orthotic has access to the lumbar adjusting mechanism while wearing the orthotic and is able to adjust the lumbar support without requiring additional tools. A third advantage is the parts of the orthotics, including the adjustment mechanism or the lumbar piece, are modular and can be removed or replace easily by any individual, again, without the use of tools. The ability to replace modular parts is highly advantageous for the market because it reduces the cost to the consumer by offering a system that can be tuned to fit the lordotic curve of the wearer without requiring the consumer to purchase a new, complete, and customized orthotic. The lumbar piece can be adjusted and/or replaced to fit the length, depth, vertical position, or other characteristic of the lordotic curve.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible maimer consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. An orthotic for providing lumbar support to a wearer, comprising:
a back panel and at least one additional member that cooperate to girdle a lower torso region of the wearer;
a flexible lumbar piece mechanically coupled to the back panel; and
an adjusting member that is operatively positioned to alter bowing of the lumbar piece relative to the back panel.

2. The adjustment system of claim 1, wherein the lumbar piece comprises a metal material.

3. The adjustment system of claim 1, wherein the lumbar piece comprises a plastic material.

4. The adjustment system of claim 1, wherein the lumbar piece is removable from the orthotic.

5. The adjustment system of claim 1, wherein the lumbar piece has an effective length of at least 5 cm.

6. The adjustment system of claim 1, further comprising a series of detents that operate to provide discrete positions for the adjusting member.

7. The adjustment system of claim 1, wherein the adjusting member further comprises a rack and pinion.

8. The adjustment system of claim 1, wherein the adjusting member comprises a dial.

9. The adjustment system of claim 1, wherein the adjusting member comprises a lever.

10. The adjustment system of claim 1, wherein the adjusting member is positioned below the lumbar piece.
